(19) United States
(12) Patent Application Publication
(10) Pub. No.: US 2026/0077122 A1
(43) Pub. Date: Mar. 19, 2026

(54) INFUSION DEVICES AND ASSOCIATED METHODS

(71) Applicant: Unomedical A/S, Lejre (DK)

(72) Inventors: Erik Othel-Jacobsen, Snekkersten (DK); Henrik Tipsmark, Roskilde (DK); Rune Frimand Byager, Hvalsoe (DK); Petros Malliotakis, Lejre (DK); Jesper Erichsen, Roskilde (DK)

(21) Appl. No.: 19/393,930

(22) Filed: Nov. 19, 2025

Related U.S. Application Data
(63) Continuation of application No. PCT/EP2024/063787, filed on May 17, 2024.
(60) Provisional application No. 63/503,284, filed on May 19, 2023.
(60) Provisional application No. 63/503,328, filed on May 19, 2023.

(30) Foreign Application Priority Data
Jul. 6, 2023 (GB) 2310436.7
Jul. 6, 2023 (GB) 2310437.5

(51) Int. Cl. A61M 5/158; A61M 5/142

(52) U.S. Cl. CPC A61M 5/158; A61M 2005/14252; A61M 2005/1585; A61M 2205/0266

(57) ABSTRACT

An infusion device comprising a cannula and a housing for containing the first cannula. The cannula is movable from a retracted position within the housing to an extended position in which a distal portion of the cannula extends from the housing for insertion into soft tissue of a patient and to form an anchor therein for securing the infusion device to an infusion site.

### TECHNICAL FIELD

The invention relates to infusion devices and associated methods. Particularly, though not exclusively, the invention relates to infusion devices for infusing a therapeutic agent, for example insulin, into soft tissue of a patient.

### BACKGROUND

For patients with diabetes, insulin therapy is often an important part of their treatment, helping to regulate blood sugar levels and store excess glucose for energy. There are two principal modes for delivering insulin. The first mode includes syringes and injector pens, which are used to inject doses of insulin typically three to four times a day, depending on, inter alia, the type of diabetes and blood sugar levels of the patient. While these devices are simple and low cost, delivering each dose of insulin requires a needle stick. The second mode uses an infusion pump, sometimes called an insulin pump, which delivers controlled doses of insulin throughout the day. An infusion pump can be used to deliver insulin to a patient continuously (basal dose), on demand (bolus dose) or at scheduled intervals. Infusion pumps are more complex and expensive than syringes and pens, though enable improved regulation of blood sugar levels, for example by programmable delivery schedules, and fewer needle sticks. The second mode is known as continuous subcutaneous insulin infusion (CSII) therapy.

Infusion pump systems for CSII therapy may be worn by the patient. The systems typically comprise a combined infusion pump and reservoir for containing an insulin drug, for example human insulin or analogue insulin, and an insulin infusion set. The infusion set may comprise a cannula (for example, a polymeric catheter or metal needle) for insertion subcutaneously into the patient and flexible tubing for fluidly connecting the cannula to the reservoir. Once the cannula is inserted into the patient, it may remain in place for a period of time, i.e. days, to allow for continuous delivery of the insulin drug. The current recommended wear time for insulin infusion sets is two to three days, to avoid problems that may arise relating to the infusion set itself or to the infusion site. However, such problems may still arise within recommended wear times, resulting in early removal of the infusion set and more frequent site rotation across infusion sites, for example the buttocks, abdomen and arms.

While problems relating to the infusion set have been well investigated and addressed in recent years, there remains little understanding and few solutions to address problems relating to the infusion site. Problems relating to the infusion site include pain, bleeding, infection, skin irritation, erythema, lipohypertrophy and lipoatrophy. Problems at the infusion site may lead to the build-up of scar tissue, which consequently lowers insulin sensitivity and increases the risk of hypoglycaemia, as well as having a cosmetic impact on patients. All these problems can deter patients from continuing to use their infusion pumps, resulting in poorer patient outcomes.

It is known that problems at the infusion site are a consequence of the immune response to the presence of the cannula and the insulin drug in the body. The immune system responds by activating and progressing the foreign body reaction (FBR)—an inflammatory and fibrotic process that occurs upon introducing a foreign material into the body. In FBR, cells of the immune system identify foreign material and attempt to degrade it, or otherwise encapsulate the material by forming a physical barrier to isolate it from the rest of the body. FBR is a problem for increasing wear times of infusion sets in CSII therapy, as the immune system reacts to both the cannula and the insulin drug. This limitation prevents realising the full potential of CSII therapy.

The problem presented by FBR is particularly acute in the development of so-called closed-loop systems. In closed-loop systems, an infusion pump system is used in combination with a continuous glucose monitoring (CGM) device, to continually monitor blood sugar levels and adjust the amount of insulin delivered to the patient automatically. CGM devices are already achieving 14-day wear times, and are thus currently achieving superior wear times compared to infusion sets.

A further problem can be achieving and maintaining correct placement of the cannula in the infusion site. When delivering insulin, it is desirable that it be delivered into the hypodermis, also known as the subcutaneous facia, in order to be optimally adsorbed. Insulin absorption and action in the hypodermis is more consistent than when insulin is delivered intramuscularly. The hypodermis is approximately 3 to 4 mm below the surface of the skin, under the epidermis and the dermis, and the thickness of the hypodermis may be as little as 1 mm or less. Placing a cannula into the soft tissue of a patient so that the tip is correctly located and maintained in hypodermis can be difficult, and incorrect placement can have undesirable effects, for example absorption may occur to quickly, meaning the therapeutic effect may not last as long as intended, and delivery may be painful.

It is an object of embodiments of the invention to provide an improved infusion device that attempts to circumvent FBR, increase wear times of infusion sets, improve placement of the cannula in the infusion site and/or at least mitigate one or more problems associated with known arrangements.

### SUMMARY OF THE INVENTION

According to an aspect of the invention, there is provided an infusion device comprising: a cannula; and a housing for containing the cannula, wherein the cannula is movable from a first retracted position within the housing to an extended position in which a distal portion of the cannula extends from the housing for insertion into soft tissue of a patient and wherein the cannula forms a coil when within the housing. This arrangement may help to improve the placement of the cannula and/or the ease with which the cannula is inserted in the soft tissue of the patient. This arrangement may help to reduce or inhibit FBR.

In certain embodiments, the housing may comprise first and second portions rotatably engaged with one another and the cannula is moveable from the first retracted position to the extended position by a first relative rotation of the first and second portions that rotates the cannula about a longitudinal axis of the coil. Optionally, the first and second portions may be rotatably engaged with one another by a screw thread and the first relative rotation moves the cannula axially. Additional, or alternatively, proximal end of the cannula may be fixedly engaged with the second portion and thereby the cannula and the second portion may be rotatable about the longitudinal axis of the coil together as one. The coil may comprise at least two complete turns in the retracted position.

In certain embodiments, the cannula may be elastically deformed to form the coil in the first retracted position and thereby a resiliency of the cannula biases the coil to uncoil. Optionally, the cannula may be elastically deformed such that the resiliency of the cannula biases adjacent turns of the coil apart from one another to increase a spacing between the adjacent coils as the cannula moves from the first retracted position to the extended position. Additionally, or alternatively, the cannula may be elastically deformed such that the resiliency of the cannula biases a diameter of the coil to increase as the cannula moves from the first retracted position to the extended position.

In certain embodiments, the cannula may be substantially straight when extending from the housing. Alternatively, the cannula may form the coil when extending from the housing. The coil may comprise at least two complete turns in the extended position.

In certain embodiments, the cannula is moveable from the extended position to a second retracted position by a second relative rotation of the first and second portions that rotates the cannula about the longitudinal axis of the coil. Additionally, or alternatively, the housing may comprise a guide for guiding the distal portion of the cannula as the cannula moves from the first retracted position to the extended position. The cannula may be deflected by the guide as the cannula moves from the first retracted position to the extended position, which may help to achieve a desired insertion angle.

In certain embodiments, the infusion device may comprise an insertion aid for selective engagement with the second portion to provide a finger grip for aiding a user to make the relative rotation.

In certain embodiments, the cannula may comprise, or be formed of, a superelastic material. The superelastic material may be a nickel titanium alloy, i.e. nitinol, and optionally wherein the nickel titanium alloy has an austenite finish temperature within a range of −15° C. to 20° C. The cannula may have a wall thickness of at least 20 μm and/or no greater than 400 μm. Additionally, or alternatively, the cannula may have an outer diameter of at least 0.4 mm and/or no greater than 0.7 mm, and/or the cannula may have an inner diameter of at least 0.2 mm and/or no greater than 0.5 mm.

According to another aspect of the invention, there is provided a method of inserting a cannula into soft tissue of a patient, the method comprising the steps of: providing an infusion device according to any proceeding claim; and moving the cannula from a first retracted position within the housing in which the canula forms a coil to an extended position in which a distal portion of the cannula extends from the housing and is inserted into the soft tissue. This method may help to improve the placement of the cannula and/or the ease with which the cannula is inserted in the soft tissue of the patient.

According to another aspect of the invention, there is provided an infusion device comprising: a first cannula; and a housing for containing the first cannula, wherein the first cannula is movable from a retracted position within the housing to an extended position in which a distal portion of the first cannula extends from the housing for insertion into soft tissue of a patient and to form an anchor therein for securing the infusion device to an infusion site. Securement inhibits lifting of the device from the infusion site. This arrangement may help to improve the placement of the cannula and/or the ease with which the cannula is inserted in the soft tissue of the patient. This arrangement may help to reduce or inhibit FBR. At least a portion of the distal portion may be inclined with respect the skin of the patient/base of the device. Removal of the device may require retraction of the needle, i.e. movement of the needle from the extended position to the retracted position.

In certain embodiments, the infusion device may comprise second and third cannulas movable according to the first cannula. Optionally, the first, second and third cannulas may be positioned equidistant from one another. Additionally, or alternatively, the infusion device may comprise a common support upon which each of the first, second and third cannulas is supported.

In certain embodiments, at least the first cannula may form a coil when extending from the housing. Alternatively, at least the first cannula may forms a hook when extending from the housing. The second and/or third cannulas may form a respective coil or hook when extending from the housing.

In certain embodiments, the first, second and third cannulas may extend away from one another when extending from the housing. At least one of the first, second and third cannulas may be substantially straight when extending from the housing. The infusion device may be free from adhesive means for securing the infusion device to an infusion site.

In certain embodiments, the at least the first cannula may comprise, or be formed of, a superelastic material. The superelastic material may be a nickel titanium alloy, i.e. nitinol, and optionally wherein the nickel titanium alloy has an austenite finish temperature within a range of −15° C. to 20° C. At least the first cannula may have a wall thickness of at least 20 μm and/or no greater than 400 μm. Additionally, or alternatively, at least the first cannula may have an outer diameter of at least 0.4 mm and/or no greater than 0.7 mm, and/or at least the first cannula may have an inner diameter of at least 0.2 mm and/or no greater than 0.5 mm.

According to another aspect of the invention, there is provided a method of securing an infusion device to an infusion site, the method comprising the steps of: providing an infusion device according to any proceeding claim; and moving the first cannula from a retracted position within the housing to an extended position in which a distal portion of the first cannula extends from the housing and is inserted into soft tissue of a patient, the first cannula forming an anchor therein to secure the infusion device to the infusion site.

According to another aspect of the invention, there is provided an infusion device comprising: first and second cannulas; and a distributor member comprising a fluid pathway extending therethrough, the distributor member being operable to selectively fluidly connect each of the first and second cannulas to the fluid pathway. This arrangement may help to reduce or inhibit FBR.

In certain embodiments, the distributor member may be operable to selectively fluidly connect each of the first and second cannulas to the fluid pathway non-simultaneously. Additionally, or alternatively, the distributor member may be rotatable about an axis of rotation to selectively fluidly connect each of the first and second cannulas to the fluid pathway, i.e., the fluid pathway may rotate with/as part of the distributor member. Optionally, the distributor member may be rotatable in one direction only.

In certain embodiments, the infusion device may comprise first and second cannula hubs each comprising a respective inlet port fluidly connected to the first and second cannulas respectively and the distributor member may comprise an outlet port at which the fluid pathway terminates engageable with each of the inlet ports. Optionally, the distributor member may be rotatable to bring each of the inlet ports into coaxial alignment with the outlet port. Optionally, the distributor member may be axially moveable between a non-fluidly connecting position and a fluidly connecting position. Optionally, the distributor member may be biased from the non-fluidly connecting position to the fluidly connecting position. The distributor member may be moveable to the fluidly connecting position only when one of the inlets ports is in coaxial alignment with the outlet port. The outlet port may be receivable within the inlet ports, or vice versa, when in the fluidly connecting position. Optionally, each of the inlet ports may be a frustoconical recess formed in the respective cannula hub and/or the outlet port may be a frustoconical protrusion, which may be complementary to each of the recesses.

In certain embodiments, the infusion device may comprise a common support upon which each of the first and second cannulas is supported. The distributor member may be supported upon the common support.

In certain embodiments, the infusion device may comprise a third cannula and the distributor member may be operable to selectively fluidly connect the third cannula to the fluid pathway. The first, second and third cannulas may be positioned equidistant from one another. Optionally, the distributor member may comprise a pointer to indicate a rotational position of the outlet port. The distributor member may comprise a body and the pointer may extend radially from body. The outlet may be formed in the pointer.

In certain embodiments, the infusion device may comprise first and second cannula hubs comprising: the first and second cannulas, respectively; and a respective housing for containing the cannula, wherein each cannula is movable from a first retracted position within the respective housing to an extended position in which a distal portion of the cannula extends from the housing for insertion into soft tissue of a patient and wherein the cannula forms a coil when within the housing.

In certain embodiments, the infusion device may comprise first and second cannula hubs comprising: the first and second cannulas, respectively; and a respective housing for containing the cannula, wherein each cannula is biased from a first retracted position within the respective housing to an extended position in which a distal portion of the cannula extends from the housing for insertion into soft tissue of a patient by a resiliency of the cannula due to the cannula being elastically deformed to form a coil when within the housing.

According to another aspect of the invention, there is provided a method of subcutaneous infusion of a therapeutic agent, the method comprising the steps of: providing an infusion device according to any proceeding claim: inserting the first and second cannulas into first and second infusion sites of a patient respectively; and delivering the therapeutic agent to the first and second infusion sites through the first and second cannulas by selectively fluidly connecting the first and second cannulas to the fluid pathway.

According to another aspect of the invention, there is provided an infusion device comprising: a cannula; and a housing for containing the cannula, wherein the cannula is biased from a first retracted position within the housing to an extended position in which a distal portion of the cannula extends from the housing for insertion into soft tissue of a patient by a resiliency of the cannula due to the cannula being elastically deformed to form a coil when within the housing. This arrangement may help to improve the placement of the cannula and/or the ease with which the cannula is inserted in the soft tissue of the patient. This arrangement may help to reduce or inhibit FBR.

In certain embodiments, the cannula may be elastically deformed such at least one turn of the coil has a greater diameter in the first retracted position than in the extended position. Additionally, or alternatively, the cannula may be elastically deformed such that a spacing between adjacent turns of the coil is greater in the extended position than in the first retracted position.

In certain embodiments, the cannula may be maintained in the first retracted position by a trigger mechanism applying a radially outward tensile force and/or an axially compressive force on the coil. Optionally, the cannula may transition from the first retracted position to the extended position by release of the trigger mechanism.

In certain embodiments, the coil may comprise at least two complete turns in the retracted position. Additionally, or alternatively, the cannula is substantially straight when extending from the housing. The cannula may form the coil when extending from the housing. The coil may comprise at least two complete turns in the extended position.

In certain embodiments, the housing may comprise first and second portions rotatably engaged with one another and the cannula may be moveable from the extended position to a second retracted position by a relative rotation of the first and second portions that rotates the cannula about a longitudinal axis of the coil. Optionally, the first and second portions are rotatably engaged with one another by a screw thread and the relative rotation moves the cannula axially. Additionally, or alternatively, a proximal end of the cannula may be fixedly engaged with the second portion and thereby the cannula and the second portion may be rotatable about the longitudinal axis of the coil together as one. A relative movement of the first and second portions may release the trigger mechanism.

In certain embodiments, the housing comprises a guide for guiding the distal portion of the cannula as the cannula is biased from the retracted position to the extended position, and optionally wherein the cannula is deflected by the guide as the cannula is biased from the first retracted position to the extended position.

In certain embodiments, the cannula may comprise, or be formed of, a superelastic material. The superelastic material may be a nickel titanium alloy, i.e. nitinol, and optionally wherein the nickel titanium alloy has an austenite finish temperature within a range of −15° C. to 20° C. The cannula may have a wall thickness of at least 20 μm and/or no greater than 400 μm. Additionally, or alternatively, the cannula may have an outer diameter of at least 0.4 mm and/or no greater than 0.7 mm, and/or the cannula may have an inner diameter of at least 0.2 mm and/or no greater than 0.5 mm.

According to another aspect of the invention, there is provided a method of inserting a cannula into soft tissue of a patient, the method comprising the steps of: providing an infusion device according to any proceeding claim; and releasing the cannula from a first retracted position within the housing in which the cannula is elastically deformed to form a coil to an extended position in which a distal portion of the cannula is biased to extend from the housing by a resiliency of the cannula and is inserted into the soft tissue.

It is to be understood that each of the above-described aspects of the invention may be combined with one or more of the other aspects. Similarly, any of the optional features described in relation to each of the above-described aspects may be combined with the other aspects and features described in respect thereof.

### BRIEF DESCRIPTION OF THE DRAWINGS

Embodiments of the invention will now be described, by way of example only, with reference to the accompanying figures, in which:

FIG. 1 is a perspective view of an infusion device according to an embodiment of the invention;

FIG. 2 being is an exploded view of the infusion device of FIG. 1;

FIG. 3 is a cross sectional perspective view taken through a centreline of the infusion device of FIGS. 1 and 2, showing a subset of the features;

FIG. 4 is a further perspective view if the infusion device of FIGS. 1 and 2, highlighting the locations of two inlet ports;

FIGS. 5 and 6 are cross sectional views taken through a centreline of the infusion device of FIGS. 1 and 2, showing the a cannula of the device in a retracted position and an extended position, respectively;

FIG. 7 is a graph of the temperature-induced phase transformation of a nitinol alloy between austenitic and martensitic phases;

FIG. 8 is a graph of the relationship between the martensite start temperature Mₛ and the composition of nitinol for a range of nitinol alloys;

FIG. 9 is a perspective view of an infusion device according to another embodiment of the invention, the device being in a non-fluidly connecting condition;

FIG. 10 is a top view of the infusion device of FIG. 9;

FIG. 11 is a cross sectional perspective view taken through a centreline of the infusion device of FIG. 9, highlighting a fluid pathway;

FIG. 12 is a side view of the infusion device of FIG. 9, the device being in a fluidly connecting condition;

FIGS. 13 and 14 are cross sectional views taken through a centreline of the infusion device of FIG. 9, showing a distribution member in alignment and in non-alignment with a cannula hub, respectively; and

FIGS. 15 to 17 are cross sectional perspective views taken through a centreline of an infusion device according to another embodiment of the invention.

### DETAILED DESCRIPTION

Embodiments of the invention have particular application for use with infusion pump systems such as an infusion pump for delivery of a therapeutic agent, the infusion pump comprising a fluid pump and a reservoir, and an infusion set having a cannula and tubing for connecting the cannula to the reservoir. However, other applications are contemplated, for example cannula access ports and alternative infusion pump systems such as patch pumps, which have an internal reservoir and therefore no tubing. In certain embodiments, the infusion pump may be an insulin pump for CSII therapy and the therapeutic agent may be an insulin drug, for example human insulin or analogue insulin. The therapeutic agent may be an insulin solution. Embodiments of the invention are intended for delivering insulin to a patient at a single infusion site or multiple infusion sites over an extended period of time. Used herein, an extended period of time is to be understood to mean at least four days. More specifically, an extended period of time may include four to seven days, seven or more days, seven to 10 days, and 10 or more days. An extended period of time may include 14 or more days. Embodiments of the invention may be used in closed loop systems, wherein the extended period of time may be at least equal to the wear time and/or the lifetime of a CGM device.

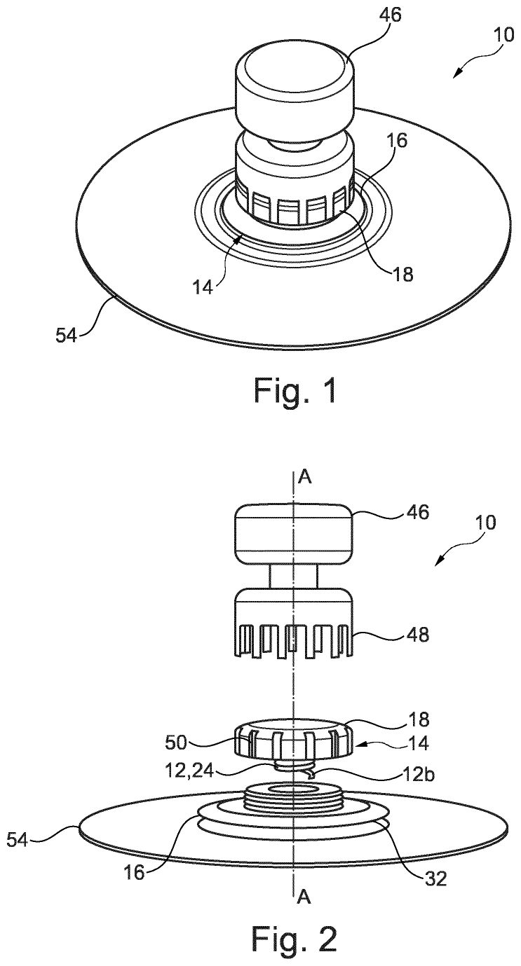

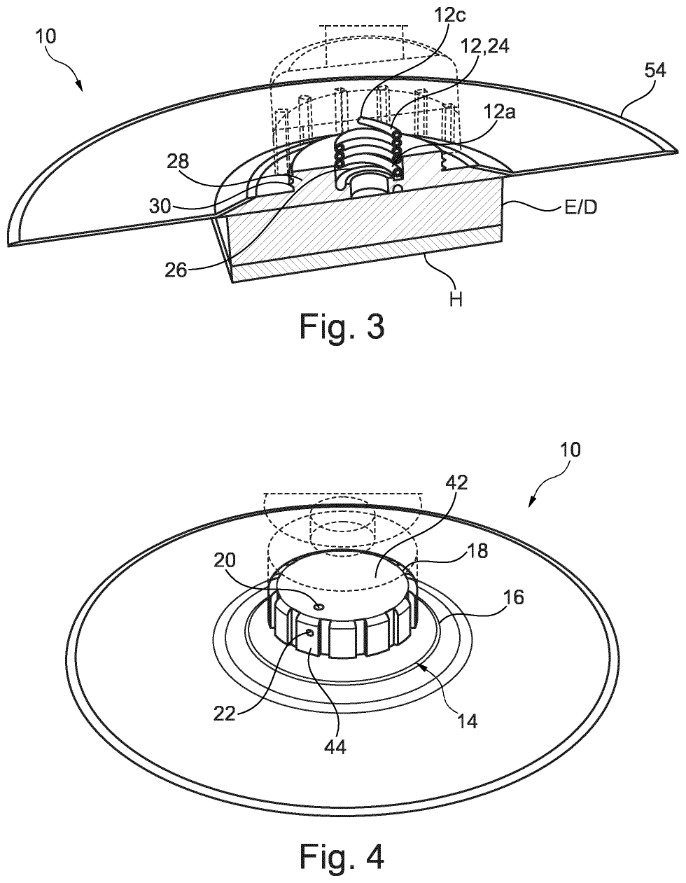

FIGS. 1 to 6 illustrate an infusion device 10 according to an embodiment of the invention, which may be referred to as a cannula hub or infusion hub. The cannula hub 10 comprises a cannula 12 and a housing 14 for containing the cannula 12. The housing 14 comprises a first portion 16, hereafter referred to as a body, and a second portion 18, hereafter referred to as a head. The head 18 comprises first and second inlet ports 20, 22 (best shown in FIG. 4) each in fluid communication with the cannula 12, and each of the inlet ports 20, 22 may be configured for attachment to a length of tubing (not shown) for delivering a therapeutic agent to the cannula hub 10 and subsequently to the soft tissue of a patient, i.e. an infusion or delivery site, via the cannula 12.

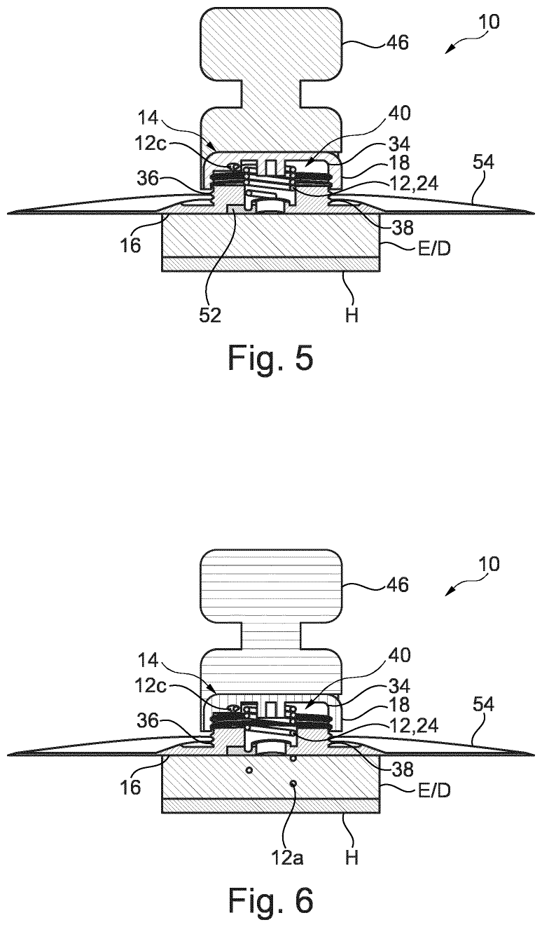

The cannula 12 is movable from a retracted position within the housing 14 to an extended position in which a distal portion 12a of the cannula 12 extends from the housing 14 for insertion into the soft tissue of the patient. To aid insertion, the distal end 12a of the cannula 12 may comprise a sharp tip 12b, for example a lancet-type tip, for cutting soft tissue. FIG. 5 shows the cannula 12 in the retracted position in which the cannula 12 forms a coil 24 within the housing 14. FIG. 6 shows the cannula in the extended position in which the cannula 12 continues to form the coil 24 extending from the housing 14, though this need not be the case in certain embodiments as is explained below. The cannula 12 extends from what may be considered an underside of the housing 14 and/or the body 16, exiting the housing 14 though an opening in the underside. The underside is a patient-facing side of the housing 14 and/or body 16. Within the housing 14, the cannula 12 is at least partially contained and/or supported within the body 16. To this end, the body 16 may comprise a central space or chamber 26 in which the coil 24 is at least partially contained. In the illustrated embodiment, the chamber 26 is cylindrical and is delimited by a circumferentially, or otherwise peripherally, extending wall 28. However, in other embodiments, the chamber 26 may be any suitable shape, including cuboidal or other polyhedral prismatic shapes. The wall 28 may be suitably configured to provide the desired shape of the chamber 26. Additionally, or alternatively, the wall 28 may provide a radially outward facing surface 30 of the body 16. The surface 30 may cylindrical. The body 16 may further comprise an annular flange 32 extending circumferentially about the wall 28 and forming part of the underside of the body 16. The flange 32 may help to improve the stability of the cannula hub 10 when attached to the skin of the patient.

The body 16 may be rotatably engaged with the head 18 such that the cannula 12 is moveable from the retracted position to the extended position by a relative rotation of the body 16 and the head 18, as explained in more detail below. The relative rotation causes the cannula 12 to rotate about a longitudinal axis of the coil 24, which in the illustrated embodiment is coincident with a longitudinal axis A-A of the cannula hub 10. Therefore, the centre of rotation of the relative rotation may lay on the longitudinal axis A-A of the cannula hub 10, i.e. the head 18 may rotate about the longitudinal axis of the coil 24 and the longitudinal axis A-A of the cannula hub 10 during the relative rotation. This provides for a simple and economic arrangement of parts. However, this arrangement need not be present in other embodiments.

The body 16 and the head 18 may be rotatably engaged with one another by a screw thread. More specifically, the radially outward facing surface 30 of the body 16 and a radially inwardly facing surface 34 of the head 18 may comprise complementary and cooperating threads 36, 38, respectively. As a consequence of being threadably engaged with one another, the body 16 may be at least partially receivable within the head 18. Though it is contemplated that the body 16 may be at least partially receivable within the head 18 when otherwise engaged therewith. To this end, the head 18 may comprise a void or space 40 in which the head 18 is at least in part receivable. The space 40 is provided in what may be considered an underside of the head 16, and the head 18 may be received over the body 16. In use, much of the body 14, or at least the wall 28, may consequently be hidden from view. As best shown in FIGS. 5 and 6, the cannula 12 forming the coil 24 extends from the chamber 26 of the body 16 and into the space 40 of/under the head 18.

As above, the head 18 comprises first and second inlet ports 20, 22 each in fluid communication with the cannula 12. However, in certain embodiments, only one of the first and second inlet ports 20, 22 may be provided. Alternatively, both of the inlet ports 20, 22 may be present and one or both may be selectively provided with a closure member. As shown in the accompanying figures, the first inlet port 20 is provided in what may be considered an upwardly facing surface 42 of the housing 14 and/or the body 16. The upwardly facing surface 42 is an opposite surface of the underside, i.e. the patient-facing side, of the housing 14 and/or the body 16. Also shown is the second inlet port 22 being provided in radially outward facing surface 44 of the head 18.

The head 18 further comprises a channel (not shown) terminating at the first and second inlet ports 20, 22—the channel may comprise two branches in embodiments having both of the inlet ports 20, 22—and extending through the head 18 and into the space 40 for fluidly connecting to the cannula 12. A proximal end 12c of the cannula 12 may be received within the channel in manner forming a fluid connection between the cannula 12 and the channel, for example the cannula 12 may form a fluidly sealing push fit within the channel. However, any suitable means of fluidly connecting the cannula 12 and the channel may be provided, for example via a connector connected to the proximal end 12c of the cannula 12. The connection between the cannula 12 and the head 18 may fixedly engage each with the other such that the cannula 12 and the head 18 are rotatable about the longitudinal axis of the coil 24 together as one. In other words, the connection may transmit rotation of the head 18, applied by a user, directly to the cannula 12.

In embodiments having threaded engagement of the body 16 and the head 18, as the user rotates the head 18 the coil 24 may rotate within the housing 14 about the longitudinal axis A-A while simultaneously moving axially in a distal direction. This rotation and movement of the coil 24 advances the canula 12 from the retracted position towards the extended position and, in use, into the soft tissue of the patient. To facilitate rotation of the head 18, the cannula hub 10 may comprise, or be supplied with, an insertion aid 46. Since the cannula hub 10 may be designed to be as small and discrete as possible, some users may find it difficult grip the head 18 effectively in order to make the required rotation to move the cannula 12 to the extended position. As such, the insertion aid 46 may be suitably configured for selective engagement with the head 18 to provide a finger grip—providing an increased surface area for the user to grip relative to the head 18—for aiding the user to make the relative rotation. As shown in the accompanying figures, the insertion aid 46 may comprise one or more fingers 48 for engaging complementary and cooperating slots 50 in the head 18. This engagement transmits a torque applied to the insertion aid 46 by the user to the head 18 to make the relative rotation. However, as the skilled reader will appreciate, many other arrangements may suitably provide such engagement, for example the engaging parts may take the form of hex driver arrangement.

In certain embodiments, the housing 14 may comprise a guide 52 for guiding the distal portion 12b of the cannula 12 as the cannula 12 exits the housing 14 and thereby moves from the retracted position to the extended position. The guide 52 may comprise a tunnel or passage, through which cannula 12 passes before exiting the housing 14 through the opening in the underside thereof. As such, the guide 52 may comprise the opening and/or form part of the underside of the housing 14. The guide 52 may restrict or control displacement of the cannula 12 from its intended path, which may facilitate providing a desired insertion angle into the soft tissue of the patient. The guide 54 may help the cannula 12 to follow a path of the coil 24 as the cannula extends from the housing 14. However, in certain embodiments, the guide 52 may cause the cannula 12 to be deflected as exits the housing 14 and moves from the first retracted position to the extended position. This may be achieved by providing the tunnel with a bend or similar, and may facilitate providing the desired insertion angle into the soft tissue of the patient. A suitable insertion angle is, for example, 30° relative to the skin of the patient. Suitable insertion angles include insertion angles from 10° and/or up to 90°. The guide 52 may be formed integrally as part of the housing 14 and/or the body 16, or may be a distinct part attached thereto.

The cannula 12 may be elastically deformed to form the coil 24 in the retracted position. In other words, when the cannula 12 is in the retracted position it may be subject to an applied force that is actively one or more of stretching, twisting, compressing and bending the cannula 12, and the cannula 12 will return to an undeformed state once the force is removed. As such, an inherent resiliency of the deformed cannula 12 may bias the coil 24 to uncoil. The applied force may be applied, or at least maintained, by physical constraints of the housing 14, for example dimensions of the chamber 26 and/or the space 40 within the housing 14 provided by the head 18. The constraints may apply axially or radially acting forces on the cannula when the cannula 12 is contained within the housing 14. The constraints may change as the cannula 12 moves from the retracted position to the extended position, for example, in embodiments having threaded engagement of body 16 and the head 18, the relative rotation will draw the body 16 and the head 18 toward one another, thereby decreasing a the space 40 in an axial direction. This may maintain a level of deformation of the coil 24 as the cannula 12 moves from the retracted position to the extended position.

The cannula 12 may be undeformed or partially undeformed in the extended position. In moving the cannula 12 from the retracted position to the extended position, the applied force may be removed or reduced, for example by the constraints of the housing 14 being removed as the distal end 12a of the cannula 12 extends therefrom. As above, the cannula 12 may be deformed in different ways, and the precise way in which the cannula 12 is deformed in the retracted position will depend on the undeformed shape of the cannula 12. The undeformed shape may be a desired shape of the cannula 12 when embedded in the soft tissue of the patient, for example to provide a desired penetration depth and/or insertion angle.

In certain embodiments, the undeformed shape of the cannula 12 may be substantially straight. As such, the cannula 12 may form the coil 24 only when deformed, i.e. not when undeformed. Consequently, the cannula 12 may form the coil 24 only when contained within the housing 14. In such embodiments, the cannula 12 may be substantially straight when extending from the housing 14. However, it is contemplated that the cannula 12 may be curved when extending from the housing 14, though not necessarily forming the coil 24, for example the cannula 12 may form a J-shape when extending from the housing 14.

Alternatively, in certain embodiments, the cannula 12 may form the coil 24 both when deformed and undeformed. As such, the cannula 12 may form the coil 24 both when contained within the housing 14 and extending therefrom. In such embodiments, one or more dimensions of the coil 24 may change as the coil 24 moves from the retracted position to the extended position, for example a spacing between adjacent turns of the coil 24 may increase as the cannula 12 moves from the retracted position to the extended position, by the inherent resiliency of the cannula 12 biasing the adjacent turns apart from one another. Additionally, or alternatively, a diameter of the coil 24 may increase as the coil 24 moves from the retracted position to the extended position.

In the illustrated embodiment, the coil 24 comprises five complete turns and one incomplete turn, and has the same number of turns in both the retracted and extended positions. However, any suitable number of turns may be provided and, at least in embodiments in which the cannula 12 is elastically deformed to form the coil 24 in the retracted position, the number of turns may differ over the length of the coil 24 between the retracted and extended positions. In certain embodiments, the coil 24 may comprise at least one complete turn in the extended position and/or the retracted position. Additionally, or alternatively, the coil 24 may comprise at least two, three or more complete turns in the extended position and/or the retracted position, and may further comprise a portion of an incomplete turn. It is to be understood that the number of turns of the coil 24 refers to the total number of turns over the length of the coil 24, regardless of whether the distal portion 12a of the cannula 12 is extending from the housing 14.

The length of the cannula 12 may be selected to achieve a desired insertion depth. As described above, the skin is made up of layers. Specifically, the skin comprises three layers: the epidermis and the dermis E/D (represented as a single layer in the accompanying figures), and the hypodermis H. When delivering the therapeutic agent, it is desirable that it be delivered into the hypodermis H. As the combined thickness of the epidermis and the dermis E/D is approximately 3 to 4 mm, the length of the cannula 12 must be such that the cannula 12 may extend to at least this depth and into the hypodermis H. While the thickness of the hypodermis vary over the body, as well as between patients, and can be up to approximately 3 cm, for example over the buttocks, correct placement of the cannula 12 may be critical to ensure that the tip 12b is located in the hypodermis H for delivering the therapeutic agent. The window for correct placement of the tip 12b may be approximately 1 to 2 mm. An advantage of the cannula 12 forming the coil 24, or being otherwise curved, in the extended position, is that it increases the length of the cannula 12 embedded in the infusion site compared to a straight cannular. This may improve placement and/or securement of the cannula 12 in the soft tissue of the patient. Moreover, by the cannula 12 forming the coil 24, the cannula 12 may provide a damping effect, whereby movement of the cannula hub 10 is not transmitted to the tip of the cannula, which may improve maintenance of the correct placement, and/or an increased length of the cannula 12 may be located in the hypodermis H. The latter effect can be beneficial since it allows for tip designs having openings in wall of the cannula 12 that may improve drug delivery pressure. In certain embodiments, there may be provided variants having different cannula lengths to accommodate differences in the in the desired insertion depth.

In the illustrated embodiment, the cannula hub 10 further comprises an adhesive patch 54 for securing the cannula hub 10 to a patient's skin, and thereby to the infusion site. The patch 54 may be attached to and extend circumferentially about the housing 14 and/or the body 16. Such patches are conventional in the art. The flange 32 may help to attach the adhesive patch 54 to the housing 14, for example by being placed over the flange 32 such that, in use, the flange 54 is held between the patch 54 the skin of the patient. However, certain embodiments are contemplated that do not require an adhesive patch. In certain embodiments, the cannula 12 may be movable from the retracted position within the housing 14 to the extended position in which the distal portion 12a of the cannula 12 extends from the housing 14 for insertion into the soft tissue of the patient and to form an anchor therein for securing the cannula hub 10 to the infusion site. In other words, the cannula 12 when embedded in the soft tissue of the patient secures the cannula hub 10 to the infusion site, and may thereby negate the need for the adhesive patch 54. This has been found to work particularly well when the cannula 12 forms the coil 24 when extending from the housing 14, i.e. when the cannula 12 forms the coil 24 when embedded in the soft tissue of the patient, for providing the anchor. As above, the cannula 12 may be J-shaped when extending from the housing, and therefore may form a hook for proving the anchor. Multi-cannula hubs (as described below) may offer improved stability and/or securement using multiple cannulas to provide multiple anchors.

In use, the user places the cannula hub 10 against the skin of the patient over a desired infusion site, as shown in FIG. 5. In embodiments having the adhesive patch 54, the user may first remove a backing paper or similar to expose an adhesive surface. The backing paper may double as a protective cover that covers the opening through which the cannula 12 extends when in the retracted position. However, in certain embodiments, a separate cover may be additionally, or alternatively, provided, which the user may remove together with, separately from or instead of the backing paper, before placing the cannula hub 10 against the skin of the patient. With the cannula hub 10 held in place over the delivery site, by either the user or by action of the adhesive patch 54, the user rotates the head 18 thereby moving the cannula from the retracted position within the housing to the extended position in which the distal portion 12a of the cannula 12 extends from the housing 14 and is inserted into the soft tissue, as shown in FIG. 6. The user may subsequently fluidly connect a length of tubing for supplying the therapeutic agent to one of the inlet ports 20, 22. With the cannula 12 embedded in the soft tissue of the patient, the cannula 12 may act to secure the cannula hub to the infusion site either alone or in combination with the adhesive patch 54, if present.

To remove the cannula 12 from the infusion site, the user may rotate the head 18 in an opposing direction to that in which it was rotated for insertion, thereby moving the cannula 12 from the extended position to position in which the cannula 12 is returned within the housing 14, for example to the retracted position or an alternative retracted position. This returns the sharp tip 12b of the cannula 12 to a safe, covered position, and allows the cannula hub 10 to be removed from the skin of the patient and be disposed of safely. In the illustrated embodiment, the direction of rotation for insertion of the cannula is clockwise, as this is most intuitive. Therefore, the direction of rotation for removal of the cannula is anti-clockwise. Of course, this need not be the case in certain embodiments, and the rotations could be reversed.

In certain embodiments, the cannula 12 may be superelastic, i.e. the cannula 12 may be formed of superelastic material, for example a superelastic alloy. Superelasticity, also referred to as pseudoelasticity, is an elastic response exhibited by certain materials to an applied stress. Superelasticity occurs when an applied stress induces an austenite to martensite phase transformation in the material and a corresponding strain, which is recoverable by removing the applied stress. Certain superelastic materials exhibit recoverable strains of up to 11%, which is significantly greater than more conventional materials. For example, 316 stainless steel (16% chromium, 10% nickel and 2% molybdenum), which is commonly used in medical applications, exhibits recoverable strains of approximately 0.5%. A superelastic alloy used in medical applications is nickel titanium alloy, commonly referred to as nitinol, as it exhibits exceptional biocompatibility. The cannula 12 may be formed of nitinol. As superelasticity is a stress-induced phase transformation from austenite to martensite, for the cannula 12 to exhibit optimum superelasticity it may be formed of so-called austenitic (or superelastic) nitinol, in which nitinol will be substantially fully austenitic, i.e. the primary crystalline structure of the alloy is austenite. Nitinol will remain substantially fully austenitic above its martensite start temperature Mₛ. This is important to note because an austenite to martensite phase transformation can be induced by cooling as well by applied stress.

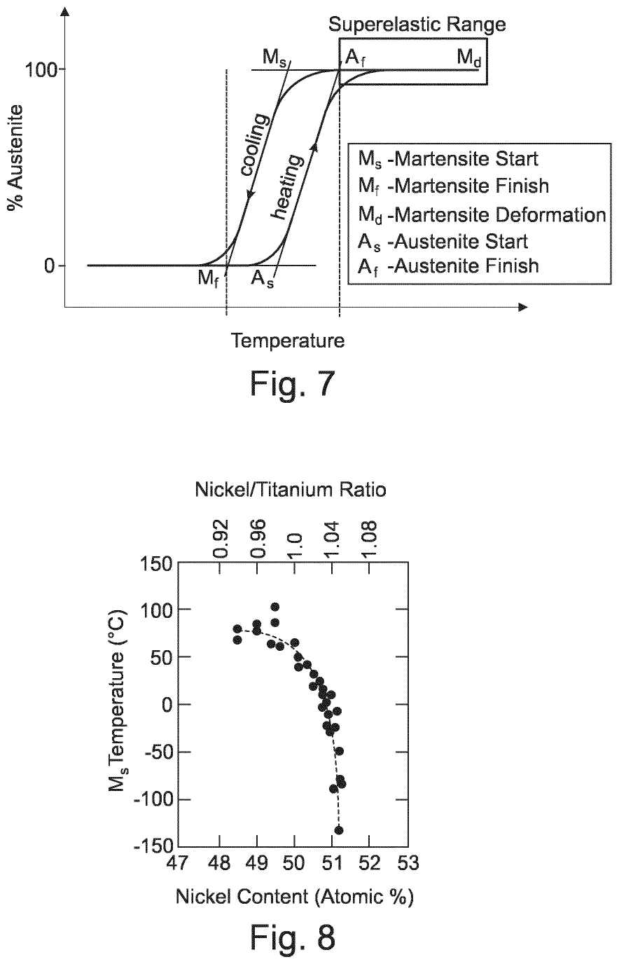

FIG. 7 shows the temperature-induced phase transformation of a nitinol alloy between the austenitic and martensitic phases, in which austenite is stable at relatively higher temperatures and martensite is stable at relatively lower temperatures. Heating nitinol beyond its austenite start temperature Aₛ causes it to transform to the austenitic phase. Nitinol will be substantially fully austenitic once heated above its austenite finish temperature A_{f}. As above, it is in this substantially fully austenitic phase that nitinol will exhibit optimum superelasticity, allowing the cannula 12 when formed of nitinol to elastically deform, i.e. flex/bend, through a relatively broad range of stresses without causing permanent deformation. The optimal superelastic range (also referred to as a superelastic window), between the austenite finish temperature A_{f} and the martensite deformation temperature M_{d}, is highlighted in FIG. 7. From the substantially fully austenitic phase, cooling nitinol beyond its martensite start temperature Mₛ causes nitinol to transition to the martensitic phase. Below its martensite finish temperature M_{f} nitinol will be substantially fully martensitic.

FIG. 7 also shows that nitinol exhibits thermal hysteresis, i.e. the temperature at which martensite transforms to austenite is not that at which austenite transforms to martensite. The hysteresis may be approximately 20 to 30° C. (i.e. A_{f}-M_{f}) for fully annealed nitinol alloys, such as those used in medical device applications. It is known that a greater thermal hysteresis will yield a greater mechanical hysteresis. The significance of its thermal hysteresis is that nitinol remains in its austenitic phase when cooled beyond its austenite finish temperature A_{f}. This means the cannula 12 when formed of austenitic nitinol will remain superelastic above its martensite start temperature Mₛ, which may therefore be the critical transformation temperature when selecting an alloy for forming the cannula 12.

FIG. 8 shows the correlation between the martensite start temperature Mₛ and the composition of nitinol. Since the martensite start temperature Mₛ is correlated with the nickel/titanium ratio it can be predetermined, for example a nitinol alloy can be selected having a martensite start temperature Mₛ from approximately −130° C. to approximately +110° C.

As above, certain embodiments of the invention may have particular application for use in devices for CSII therapy, in which the devices may be worn by a patient such that at least the tip 16 of the cannula 12 is placed in the patient's soft tissue. Due to the superelasticity of nitinol, the cannula 12 may reduce tissue damage by exhibiting a relatively high degree of flexibility. However, if the martensite percentage of the material forming the tip 16 begins to rise, exposure to stress may cause it undergo plastic deformation, resulting in an irregular, uncontrolled shape change. This may lead to tissue damage. While the patient's body heat may aid in maintaining nitinol in its austenitic phase, certain embodiments of the cannula 12 may be placed in the soft tissue of the patient to a relatively low depth, such as approximately 3 to 4 mm. As such, cold temperatures experienced by the patient may cause a temperature-induced phase transformation of the nitinol alloy forming the cannula 12 to its martensitic phase, i.e. if the martensite start temperature Mₛ is too high. Thus, in certain embodiments, the cannula 12 may be formed of a nitinol alloy selected to remain substantially fully austenitic (for example, at least 95% austenitic) in temperatures likely to be experienced by the patient to reduce the risk of the cannula 12 becoming martensitic.

As indicted in FIG. 7, superelasticity is exhibited up to the martensite deformation temperature M_{d}, which corresponds to the greatest temperature at which it is possible to stress-induce the formation of martensite. Above the martensite deformation temperature M_{d} the response to stress is non-elastic deformation of the austenitic microstructure, since martensite can no longer be formed, and thus permanent deformation. In other words, above the martensite deformation temperature M_{d} the nitinol will deform plastically and irreversibly.

The effective superelastic range of the nitinol may be increased to span more than 200° C., with a significant reduction in temperature-stress sensitivity, by subjecting the material to a controlled process. The nitinol may, for example, be subjected to an annealing process or treatment, e.g. an isobaric annealing process or treatment in Argon at approximately 350° C. to 400° C. In this way, the martensite deformation temperature M_{d} of the material is increased and the effective superelastic window or range may be increased or widened. In view of the above, the cannula 12 may be formed of a nitinol alloy having been subjected to an isobaric annealing treatment in order to increase the effective superelastic window in order to ensure that elastic deformation through a relatively broad range of stresses is possible. In this way the cannula 12 can be manufactured from nitinol that elastically bends without permanent deformation of the cannula 12.

The cannula 12 may also be formed of a nitinol alloy having an austenite finish temperature A_{f} in the range of approximately −15° C. to approximately 20° C. in order to ensure that the cannula 12 has optimal or good superelastic properties at body temperature (approximately 37° C.).

More generally, the cannula 12 may be formed of a nitinol alloy selected such that the cannula 12, or part therefore, exhibits selected properties and/or characteristics with reference to a particular temperature to be experienced by the cannula 12. In certain embodiments, the particular temperature may be body temperature (approximately 37° C.) or room temperature (for example, approximately 20 to 22° C.).

FIG. 8 also shows that nitinol is typically composed of approximately 50 to 51% nickel by atomic percent, though as shown in FIG. 4 compositions outside this range are known, and various compositions may be suitable for forming the cannula 12. In certain embodiments, an atomic ratio of nickel to titanium is between 1.01 and 1.05. In certain embodiments, an atomic ratio of nickel to titanium is between 1.02 and 1.04.

The cannula 12 being formed of austenitic or superelastic nitinol may exhibit greater flexibility than a similarly-configured cannula formed of a more conventional material, such as stainless steel. The skilled reader will understand that the range of superelasticity for a particular composition of nitinol depends largely upon its nickel/titanium ratio. In certain embodiments, the cannula 12 may be formed of nitinol having a nickel/titanium ratio selected to provide the cannula 12 with particular properties and/or characteristics. The skilled reader will also understand that the range of superelasticity of the nitinol also depends on how the material has been processed. In certain embodiments, the cannula 12 may be formed of nitinol that has been processed or treated in a controlled way in order to provide the cannula 12 with particular properties and/or characteristics.

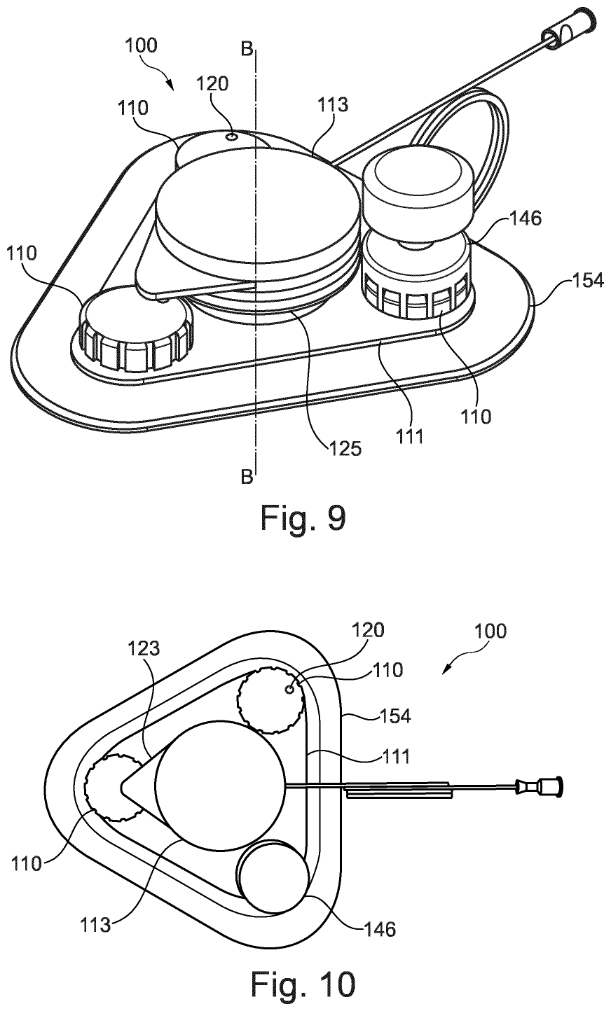

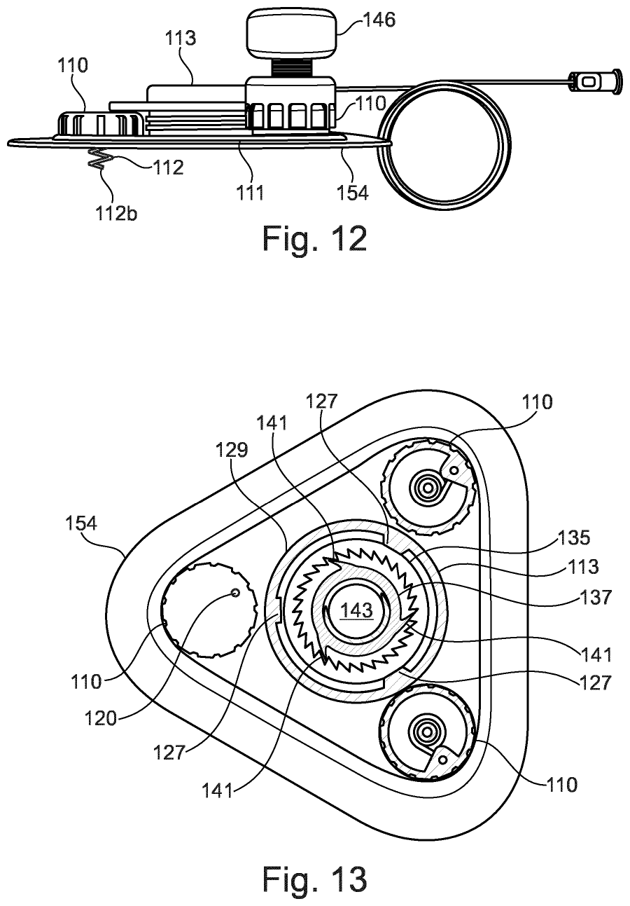

FIGS. 9 to 14 illustrate an infusion device 100 according to another embodiment of the invention, which may be referred to as a multi-cannula hub or multi-site infusion hub. The multi-cannula hub 100 comprises a plurality of cannulas 112, each of which in the illustrated embodiment is provided by one of a plurality of cannula hubs 110. Each of the cannula hubs 110 may comprise all the features and/or functionality of the cannula hub 10 described above with reference to FIGS. 1 to 8. As such, features that are the same or similar to those described above with reference to FIGS. 1 to 8 are denoted by reference numerals offset by a factor of 100. As the skilled reader will appreciate, the illustrated embodiment comprises a first cannula, a second cannula and a third cannula. However, embodiments are contemplated comprising one or more further cannulas. As shown in FIG. 12, one of the cannulas 112 is in the extended position, while the other two are in the retracted position. The cannula hubs 110 may be securable to and/or supported by a common support 111, which may maintain the cannula hubs 110 and thereby the cannulas 112 in substantially fixed relation to one another. As in the illustrated embodiment, the cannulas 112 may be spaced apart from one another equidistantly, and thereby each may lay on a respective point of an equilateral triangle. The minimum spacing between the cannulas 112 may be at least 25 mm and/or up to 40 mm, for example measured between the respective longitudinal axes of the coils 124.

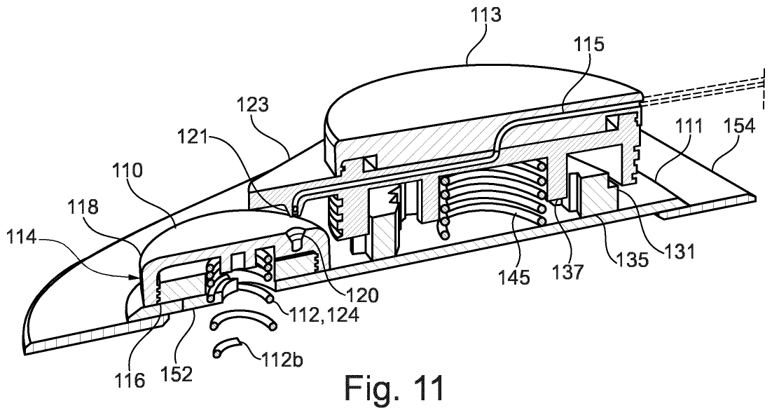

The multi-cannula hub 110 further comprises a distributor member 113. The distributor member 113 comprises a fluid pathway 115 (shown in FIG. 11) extending therethrough, and is operable to selectively fluidly connect each of the cannulas 112 to the fluid pathway 115. In certain embodiments, the distributor member 113 may be a length of tubing that is selectively fluidly connectable to at least of the respective inlet ports 120 of each of the cannula hubs 110. However, as in the illustrated embodiment, the distributor member 113 may be provided as a rotatable dial or cap, which may be securable to and/or supported by the common support 111. The distributor member 113 may be rotatable about an axis of rotation B-B to selectively fluidly connect each of the cannulas 112 to the fluid pathway 115. As shown in the accompanying figures, the distributor member 113 comprises an outlet port 121 (best shown in FIG. 11) at which the fluid pathway 115 terminates, the outlet port 121 being selectively engageable with each of the inlet ports 120 to establish a fluid connection.

In the illustrated embodiment, the outlet port 121 is formed in a pointer 123, which extends radially from a main body 125 of the distributor member 113. The function of the pointer 123 is two-fold: firstly, the pointer 123 provides a visual indication to the user as to the position of the outlet port 121 relative to the inlet ports 120: secondly, pointer 123 extends away from the main body 125 to facilitate the forming of the fluid connection between each of the cannulas 112 and the fluid pathway 115. As shown, the outlet port 121 may be formed in what may be considered an underside of the pointer 123. Consequently, the distributor member 123 may be rotatable to bring each of the inlet ports 120 into alignment, for example coaxial alignment, with the outlet port 121. The distributor member 113 may movable so as to be positionable over each of the cannula hubs 110. Successful alignment may be indicted by the relative position of the pointer 123 and one of the cannula hubs 110, with which the desired fluid connection is to be made, for example by the pointer 123 aligning with the centre of the head 118 and/or a mark or indication provided on the cannula hub 110 for the purpose of indicating alignment. With one of the inlet ports 120 in alignment with the outlet port 123, the distributor member 113 may be axially moveable between a non-fluidly connecting position (or unconnected position) and a fluidly connecting position. In other words, the distributor member 113 may be moved axially towards the cannula hub 110 to establish a fluid connection therebetween. In certain embodiments, the axial movement may introduce the outlet port 123 into the aligned inlet port 120, or vice versa, and thereby establish the fluid connection. To this end, each of the inlet ports 120 is may be frustoconical recess formed in the respective cannula hub 110 and/or the outlet port 123 may be a frustoconical protrusion complementary to each of the recesses.

FIGS. 9 and 11 best show the distributor member 113 in the non-fluidly connecting position, in which a clearance gap is present between the cannula hub 110 and distributor member 113. The gap allows the distributor member 113 to be rotated and pass over the cannula hub 110. This arrangement is the same for each of the cannula hubs 110, so that the distributor member 113 may be freely rotatable through 360° in the non-fluidly connecting position.

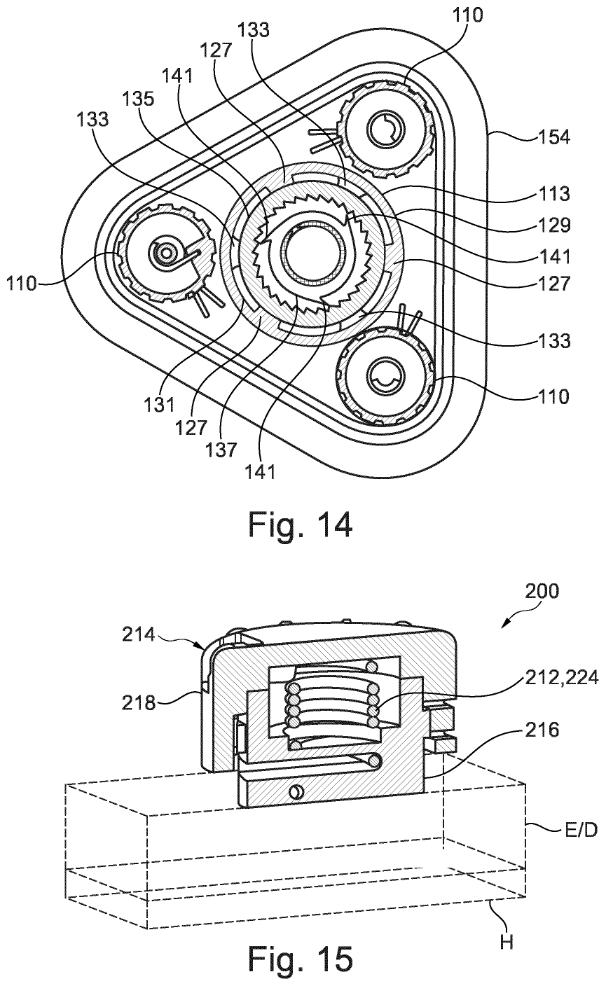

FIG. 12 best shows the distributor member 113 in the fluidly connecting position, in which the clearance gap is closed and the outlet port 123 is received within the inlet port 120 to establish the fluid connection between the cannula 112 and the fluid pathway 115. The distributor member 113 may be moveable to the fluidly connecting position only when one of the inlets ports 120 is in alignment with the outlet port 123. FIG. 13 shows inside the body 125 of the distributor member 113 in the fluidly connecting position. In contrast, FIG. 14 shows inside the body 125 in the non-fluidly connecting position. As shown in the figures, the distributor member 113 may comprise one or more radially inwardly directed projections or tabs 127 extending from a peripheral side wall 129 of the distributor member 113. With one of the inlet ports 120 in alignment with the outlet port 123, the tabs 127 are unconstrained, thereby allowing the distributor member 113 to move between the non-fluidly connecting and fluidly connecting positions. However, with none of the inlet ports 120 in alignment with the outlet port 123, the tabs 127 are constrained such that movement from the non-fluidly connecting position to the fluidly connecting position is inhibited. In the illustrated embodiment, this is achieved by an annular rail 131 upstanding from the support 111. As shown in FIG. 14, with the distribution member 113 rotatably positioned such that the outlet port 121 is between two of the cannula hubs 110, the rail 131 is aligned or axially aligned with the tabs 127 such that tabs 127 will contact the rail 131 if an attempt is made to move the distribution member 113 towards the fluidly connecting position, and thereby block movement of the distribution member 113 to the fluidly connecting position. Breaks or openings 133 may be provided in the rail 131 to allow the distribution member 113 to move into the fluidly connecting position, i.e. the openings 133 may receive and/or accommodate the tabs 127 in the fluidly connecting position, the openings 133 being aligned with rotational positions of the distribution member 113 when aligned correctly with the annular hubs 110.

By moving the distribution member 113 between the plurality of cannulas 112, the therapeutic agent may be delivered to one or more of the cannulas 112 independently, i.e. in series, thereby allowing healing at the infusion sites during intermittent non-delivery periods, in which the therapeutic agent is not delivered to the soft tissue of the patient by one or more of the cannulas 112, and thereby may reduce FBR. In use, the distribution member 113 may selectively fluidly connect one of the cannulas 112 to the fluid pathway 115 for a period of up to four days, before being moved and selectively fluidly connected one other of the cannulas 112. In certain embodiments, the period may be any suitable period, for example one of up to seven days, seven or more days, seven to 10 days, and 10 or more days. In certain embodiments the period may be one of no more than one day, one to two days and no more than two days. In use, the distribution member 113 may selectively fluidly connect one of the cannulas 112 to the fluid pathway 115 multiple times before the use of the multi-cannula hub 110 is discontinued. However, since it may be beneficial to ensure that each of the cannulas 112 is used in order, for example so that each may be provided with sufficient and/or equal non-delivery periods, the distributor member may be rotatable in one direction only. To this end, the distributor member 113 may comprise a ratchet mechanism that allows continuous rotation of the distributor member 113 in one direction while preventing rotation in the opposite direction. As shown in the illustrated embodiment, the ratchet mechanism may be provided by an annular upstanding wall 135 and a cooperating annular downwardly depending wall 137 that is partially nested within the upstanding wall 135. The upstanding wall 135 may extend from the support 111 and the downwardly depending wall 137 may extend from an underside of the distribution member 113. As in the illustrated embodiment, the upstanding wall 135 may comprise a rack of teeth 139 extending thereabout, and the downwardly depending wall 137 may have one or more pawls 141 that engage the teeth 139. The teeth 139 and pawl 141 are angled relative to one another such that when the distributor member 113 is rotated in the desired direction, the pawl moves up and over the teeth, but if an attempt is made to rotate the distributor member 113 in the opposite direction the one or more pawls 141 will catch against the first of the teeth 139 encountered, thereby locking the one or more pawls 141 thereagainst and preventing further rotation. Each of the one or more pawls 141 may be a flexible finger extending from the downwardly depending wall 137.

A void 143 delimited by the annular downwardly depending wall 137 may contain a biasing member or spring 145 that connects the distributor member 113 to the support 111. The spring 145 biases the distributor member 113 from the non-fluidly connecting position to fluidly connecting position and may help to form and/or maintain a fluid seal between the inlet ports 120 and the outlet port 121.

Additionally, or alternatively, delivery of the therapeutic agent may be divided between one or more of the cannulas, i.e. in parallel, thereby providing a relatively lower flow per delivery site by distribution to multiple sites to lower FBR under threshold levels. Accordingly, the distributor member 113 may fluidly connect the fluid pathway 115, and consequently a supply of the therapeutic agent, to one or more of the cannulas 112 either simultaneously or non-simultaneously.

In use, the user places the multi-cannula hub 100 against the skin of the patient over desired infusion sites. This may be the same as described above with reference to the cannula hub 10. As shown in the accompanying figures, the multi-cannula hub 100 may include an adhesive patch 154, though as the skilled reader will appreciate the cannulas 112 may secure the multi-cannula hub 100 to the infusion sites by providing anchors. The user inserts the cannulas 112 into the infusion sites, respectively, and delivers the therapeutic agent to the infusion sites through the cannulas 112 by selectively fluidly connecting the cannulas 112 to the fluid pathway 115.

FIG. 15 to 17 illustrate an infusion device 200 according to yet another embodiment of the invention, which may be referred to as a cannula hub or infusion hub. The cannula hub 200 may be substantially the same as, or similar to, the cannula hub 10 described above with reference to FIG. 1 to 8. The cannula hub 200 of FIG. 15 to 17 may comprise features and/or functionality of the cannula hub 10 described above with reference to FIG. 1 to 8. As such, features that are the same or similar to those described above with reference to FIG. 1 to 8 are denoted by reference numerals offset by a factor of 200. The principal difference is that the cannula 212 of the cannula hub 200 shown in FIG. 15 to 17 is biased from the retracted position within the housing 214 to the extended position by the inherent resiliency of the cannula 212 due to the cannula 212 being elastically deformed to form the coil 224 within the housing 214, i.e. stored energy within the coil 224—the stored energy being a product of the coil 224 being elastically deformed-drives movement of the cannula 212 from the retracted position to the extended position. As such, when in use, the inherent resiliency of the cannula 212 drives the cannula 212 into the soft tissue of the patient.

FIG. 15 shows the infusion device 200 in an assembled state, for example as it may be configured when supplied to the user. In the assembled state, the coil 224 may be elastically deformed or undeformed. However, if elastically deformed when in the assembled state, the coil 224 may lack sufficient elastic deformation to provide the required amount of stored energy to drive the cannula 224 into the soft tissue of the patient. The application of further deformation may therefore be needed prior to insertion of the cannula 224 to provide a suitable amount of stored energy. However, it is contemplated that in certain embodiments the coil 224 may be sufficiently elastically deformed when the infusion device 200 is in the assembled state so not to require the application of further deformation.

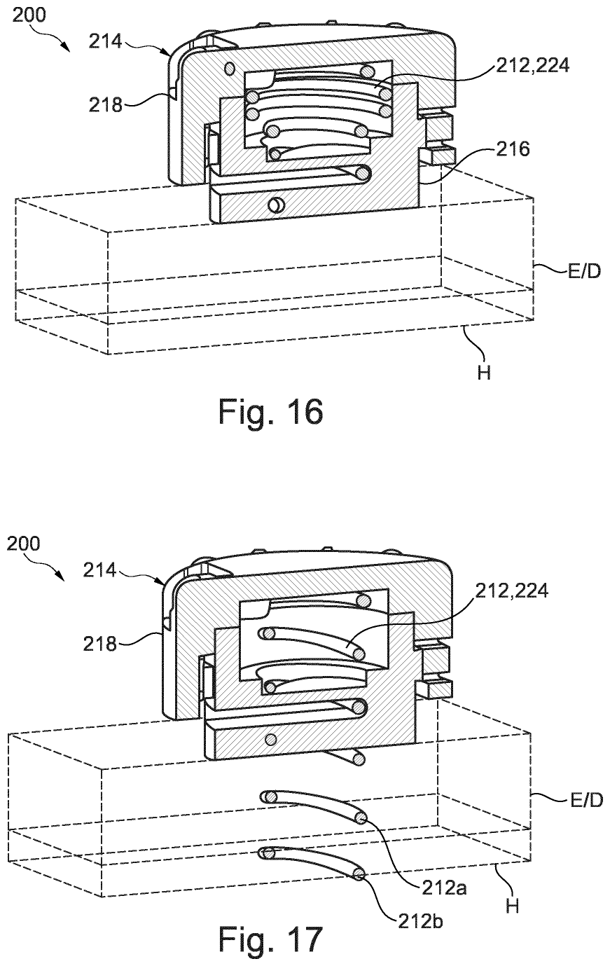

FIG. 16 shows the infusion device 200 in a primed state, for example as it may be configured immediately prior to insertion of the cannula 212 into the soft tissue of the patient. In the primed state, the coil 224 is elastically deformed. The required deformation may be applied by rotating the head 216. As described above, in relation to embodiment shown in FIG. 1 to 8, the connection between the cannula 212 the head 218 may fixedly engage each with the other. However, in the embodiment shown in FIGS. 15 to 17, movement of the distal end 212a of the cannula 212 may be inhibited, for example by a brake or clamp, such that the relative rotation of the body 216 and the head 218, rather than rotate the coil 224 about the longitudinal axis thereof, applies a torque to the coil 224 as the distal and proximal ends 212a, 212c of the cannula 212 are caused rotate relative to one another. A 360° rotation of the head 218 results in one fewer turns of the coil 224 over a larger diameter, and stores energy in the coil 224 for driving the cannula from the retracted position to the extended position and into the infusion site. In use, the user places the cannula hub 200 against the skin of the patient over desired infusion site, as described above, and releases the cannula 212 from the retracted position within the housing 214 in which the cannula is elastically deformed to form the coil 224 to the extended position in which the distal portion 212b of the cannula 212 is biased so as to extend from the housing 214 by a resiliency of the cannula 212 and is inserted into the soft tissue.

FIG. 17 shows the infusion device 200 in a deployed state, for example as it may be configured when the cannula 212 is embedded in the soft tissue of the patient. Removal may be achieved similarly to the as described above in relation to the embodiment shown in FIG. 1 to 8.

The invention is not restricted to the details of any foregoing embodiments. Throughout the description and claims of this specification, the words “comprise”, “contain”, “having” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. In particular, the phrase “certain embodiments” is to be understood to mean any embodiment described, illustrated, or otherwise disclosed herein. All of the features disclosed in this specification (including any accompanying claims, abstract and drawings), and/or all of the steps of any method or process so disclosed, may be combined in any combination, except combinations where at least some of such features and/or steps are mutually exclusive. The invention extends to any novel one, or any novel combination, of the features disclosed in this specification (including any accompanying claims, abstract and drawings), or to any novel one, or any novel combination, of the steps of any method or process so disclosed.

Attention is directed to all papers and documents which are filed concurrently with or previous to this specification in connection with this application and which are open to public inspection with this specification, and the contents of all such papers and documents are incorporated herein by reference.

1. An infusion device comprising:
a first cannula; and
a housing for containing the first cannula,
wherein the first cannula is movable from a retracted position within the housing to an extended position in which a distal portion of the first cannula extends from the housing for insertion into soft tissue of a patient and to form an anchor therein for securing the infusion device to an infusion site.

2. An infusion device according to claim 1, wherein the infusion device comprises second and third cannulas movable according to the first cannula.

3. An infusion device according to claim 2, wherein the first, second and third cannulas are positioned equidistant from one another.

4. An infusion device according to claim 2 or 3, wherein the infusion device comprises a common support upon which each of the first, second and third cannulas is supported.

5. An infusion device according to any proceeding claim, wherein the first cannula forms a coil when extending from the housing.

6. An infusion device according to any of the claims 1 to 4, wherein the first cannula forms a hook when extending from the housing.

7. An infusion device according to claims 2 to 4, wherein the first, second and third cannulas extend away from one another when extending from the housing.

8. An infusion device according to claim 7, wherein at least one of the first, second and third cannulas is substantially straight when extending from the housing.

9. An infusion device according to claim 1, wherein the infusion device is free from adhesive means for securing the infusion device to an infusion site.

10. An infusion device according to any preceding claim, wherein the cannula comprises, or is formed of, a superelastic material.

11. An infusion device according to claim 10, wherein the superelastic material is a nickel titanium alloy, and optionally wherein the nickel titanium alloy has an austenite finish temperature within a range of −15° C. to 20° C.

12. An infusion device according to any preceding claim, wherein the cannula has a wall thickness of at least 20 μm and/or no greater than 400 μm.

13. An infusion device according to any preceding claim, wherein the cannula has an outer diameter of at least 0.4 mm and/or no greater than 0.7 mm

14. An infusion device according to any preceding claim, wherein the cannula has an inner diameter of at least 0.2 mm and/or no greater than 0.5 mm.

15. A method of securing an infusion device to an infusion site, the method comprising the steps of:
providing an infusion device according to any proceeding claim; and
moving the first cannula from a retracted position within the housing to an extended position in which a distal portion of the first cannula extends from the housing and is inserted into soft tissue of a patient, the first cannula forming an anchor therein to secure the infusion device to the infusion site.

16. An infusion device comprising:
first and second cannulas; and
a distributor member comprising a fluid pathway extending therethrough, the distributor member being operable to selectively fluidly connect each of the first and second cannulas to the fluid pathway.

17. An infusion device according to claim 16, wherein the distributor member is operable to selectively fluidly connect each of the first and second cannulas to the fluid pathway non-simultaneously.

18. An infusion device according to claim 16 or 17, wherein the distributor member is rotatable about an axis of rotation to selectively fluidly connect each of the first and second cannulas to the fluid pathway.

19. An infusion device according to claim 18, wherein the distributor member is rotatable in one direction only.

20. An infusion device according to any of claims 16 to 19, wherein the infusion device comprises first and second cannula hubs each comprising a respective inlet port fluidly connected to the first and second cannulas respectively and the distributor member comprises an outlet port at which the fluid pathway terminates engageable with each of the inlet ports.

21. An infusion device according to claim 20, wherein the distributor member is rotatable to bring each of the inlet ports into coaxial alignment with the outlet port.

22. An infusion device according to claim 21, wherein the distributor member is axially moveable between a non-fluidly connecting position and a fluidly connecting position.

23. An infusion device according to claim 22, wherein the distributor member is biased from the non-fluidly connecting position to the fluidly connecting position.

24. An infusion device according to claim 22 or 23, wherein the distributor member is moveable to the fluidly connecting position only when one of the inlets ports is in coaxial alignment with the outlet port.

25. An infusion device according to claim 22, 23 or 24 wherein the outlet port is receivable within the inlet ports, or vice versa, when in the fluidly connecting position.

26. An infusion device according to claim 25, wherein each of the inlet ports is a frustoconical recess formed in the respective cannula hub and the outlet port is a frustoconical protrusion complementary to each of the recesses.

27. An infusion device according to any of claims 16 to 26, wherein the infusion device comprises a common support upon which each of the first and second cannulas is supported.

28. An infusion device according to claim 27, wherein the distributor member is supported upon the common support.

29. An infusion device according to any of claims 16 to 28, wherein the infusion device comprises a third cannula and the distributor member is operable to selectively fluidly connect the third cannula to the fluid pathway.

30. An infusion device according to claim 29, wherein the first, second and third cannulas are positioned equidistant from one another.

31. An infusion device according to any of claims 20 to 30, wherein the distributor member comprises a pointer to indicate a rotational position of the outlet port.

32. An infusion device according to claim 31, wherein the distributor member comprises a body and the pointer extends radially from body and the outlet is formed in the pointer.

33. An infusion device according to any of claims 16 to 32, wherein the infusion device comprises first and second cannula hubs comprising:
the first and second cannulas, respectively; and
a respective housing for containing the cannula,
wherein each cannula is movable from a first retracted position within the respective housing to an extended position in which a distal portion of the cannula extends from the housing for insertion into soft tissue of a patient and wherein the cannula forms a coil when within the housing.

34. An infusion device according to any of claims 16 to 32, wherein the infusion device comprises first and second cannula hubs comprising:
the first and second cannulas, respectively; and
a respective housing for containing the cannula,
wherein each cannula is biased from a first retracted position within the respective housing to an extended position in which a distal portion of the cannula extends from the housing for insertion into soft tissue of a patient by a resiliency of the cannula due to the cannula being elastically deformed to form a coil when within the housing.

35. A method of subcutaneous infusion of a therapeutic agent, the method comprising the steps of:
providing an infusion device according to any of claims 16 to 34;
inserting the first and second cannulas into first and second infusion sites of a patient respectively; and
delivering the therapeutic agent to the first and second infusion sites through the first and second cannulas by selectively fluidly connecting the first and second cannulas to the fluid pathway.
